(19) United States
(12) Patent Application Publication
(10) Pub. No.: US 2002/0006910 A1
(43) Pub. Date: Jan. 17, 2002

(54) Means for allaying drunkenness, preventing and removing alcohol intoxication and hangover syndrome and a method for allaying drunkenness, preventing and removing alcohol intoxication and hangover syndrome by using this means

(76) Inventors: Dmitri Miasnikov, Los Angeles, CA (US); Aleksandr Kashlinsky, Carlsbad, CA (US)

Correspondence Address: Donna Kasseinova, 1530 N Poinsettia Pl., # 340, Los Angeles, CA, 90046, US

(21) Appl. No.: 09/859,881

(22) Filed: May 17, 2001

Related U.S. Application Data
(60) Provisional application No. 60/263,765, filed on Jan. 25, 2001.

(30) Foreign Application Priority Data
May 18, 2000 (RU) RU 2160589 C1

(51) Int. Cl. A61K 31/715; A61K 31/70; A61K 31/195; A61K 31/194; A61K 31/375

(52) U.S. Cl. 514/23; 514/19; 514/53; 514/561; 514/574; 514/464

(57) ABSTRACT

The means for allaying drunkenness, preventing and removing alcohol intoxicaiton and hangover syndrome comprises 10 to 400 mg of a component based on succinic acid, 10 to 400 mg of at least one component based on L-glutamic acid, selected from the group consisting of L-glutamic acid, sodium mono-L-glutamate, sodium di-L-glutamate, ammonium mono-L-glutamate, ammonium di-L-glutamate, potassium mono-L-glutamate and potassium di-L-glutamate, 2 to 300 mg of at least one component based on fumaric acid, selected from the group consisting of fumaric acid, sodium monofumarate, sodium difumarate, ammononium monofumarate, ammonium difumarate, potassium monofumarate, potassium difumarate, 1 to 300 mg of ascorbic acid and up to 100 mg of at least one energizer selected from the group consisting of aspartic acid, sodium aspartate, glycine, one of amino acids, sorbic acid and at least one sugar substitute selected from the group consisting of sucrose, glucose, sorbose, sorbitol, fructose, saccarine, aspartame, xylitol, sorbitol. The means is administered per os before and/or during meals, and/or after the intake of alcohol.

### CROSS-REFERENCE TO RELATED APPLICATIONS

We hereby claim the benefit under U.S.C. 119(e) of a U.S. provisional application No. 60/263765 filed on Jan. 25, 2001.

### BACKGROUND OF THE INVENTION

The present invention relates to medicine and can be used both in the foodstuffs industry, in the form of a biologically active additive to food, and in the pharmacological industry for solving the problem of lessening the severity of drunkenness, for removing alcohol intoxication and hangover, and also for lessening attraction to alcohol.

Drunkenness and especially alcohol intoxication and subsequent hangover syndrome are accompanied by the development of a number of grave subjective and objectively observable symptoms: sensation of discomfort, inadequacy of perception, headache, disturbances in the formation of long-term memory, thirst, apathy (sometimes hyperactivity), agitation which gives way to depression, disturbances in the coordination of movements, reduction of the response rate, and many other deviations, differing in seriousness, of psychological, physiological and somatic character.

Even acute alcohol intoxication and to a still grater extent chronic alcoholization bring about aggravation of many chronic diseases associated with neuroendocrinal imbalance and weakening of the immunity, there originate or start progressing new and heretofore latent diseases, resistance of the organism to the effect of loads and extreme environmental factors decreases. Disturbances of the functions of the cardiovascular system are particularly frequent and dangerous. They manifest themselves in the development of ischemic and spastic changes in the blood supply of not only the cardiac muscle but also of the brain, practically of all parenchymatous organs and extremities, up to acute cardiac deficiency phenomena. Recurrent alcohol intoxications are often a direct cause of an expressive malignant course of hypertensive disease and of diseases of the kidneys and liver, of aggravation of latent and progressing of existing diabetes mellitus.

Depending on individual sensitivity which is determined to a considerable extent by the genetically programmed set of enzymes and by their activity, till a definite threshold dose the entrance of alcohol may not be accompanied by the development of significant physiological dysfunctions and origination of dangerous pathological changes. The value of this “safe” threshold depends on the activity, first of all, of alcohol dehydrogenases, on the particular set of their isoforms and on the activity of acetaldehyde dehydrogenases, on the energy state of mitochondria and of the organism as a whole, in particular, on the presence of a sufficient amount of other energy metabolism substrates, and, finally, on the activity of antioxidant systems. In other words, on the activity of those systems which are responsible for the utilization of ethanol and products of its oxidation—acetaldehyde and acetic acid, which are formed constantly as a result of usual metabolic transformations of foodstuffs consumed by man. Exceeding of the individual threshold—intaking appreciable doses of ethanol, is accompanied by not only by a disturbance of the psychoneurological status owing to direct action of ethanol, but also by the toxic effect of growing concentrations of acetaldehyde and acetic acid. For example, acetaldehyde provokes release of cateholamines and indolamines, causes and avalanche-like activation of the peroxidic oxidation of the lipids of biological membranes, disturbs a large number of receptor processes. Excess aldehyde forms adducts with hemoglobin, with proteins of plasma of the brain and of other organs, inhibits the transfer of reducing equivalents along the respiratory chain of mitochondria. As a results, a number of pathological processes develop. In the first place, multiple hypoxic and ischemic phenomena are observed, which progress and acquire stable character as the concentration of acetaldehyde increases. In the second place, the spermatogen synthesis becomes disturbed, the motility, survival and penetrability of spermatozoids decrease, the ovicell functions and the integrity of the mitochondrial DNA of ovicells are disturbed, this leading to infertility or to the origination of mutation, as a result of which the progeny is either not viable or suffers from various pathologies differing in severity. In the third place, th synthesis of nuclear and mitochondrial DNA on somatic cells is disturbed, and this leads to the origination of organic lesions which accelerate senility of the organism and to premature acquisition of diseases typical of senile age.

In the fourth place, repeated use of alcohol causes development of stable alcohol dependence. Finally, there arises fluidization of cellular membranes, which leads to damage to and to increase in the permeability of the hematoencephalic barrier. Thereby, the entrance of toxic endogenous products to the brain increases drastically, while the threshold of toxic perception of ethanol lowers markedly. Taking into account that in the United States alone, alcohol use costs $148 billion a year in missed work and poor job performance, the barest necessity of finding means for minimizing and liquidating the consequences of alcohol consumption is quite clear.

Today, the range of available means to minimize or eliminate alcohol intoxication and alcohol withdrawal syndrome, commonly known as a hangover, is extremely limited. According to articles published in the Annals of Internal Medicine (“Alcohol Hangover” Jun. 6, 2000, Volume 132 Number 11), The New York Times (“Morning-After Pill for Hangovers?” Dec. 27, 2000), there are currently no products on the U.S. market that could minimize alcohol intoxication and prevent hangovers—or at least treat the symptoms effectively and quickly. In terms of household-level remedies, there are, of course, ample hors d'oeuvres, which lessen the ethanol entrance rate and facilitate oxidation of ethanol products to carbon dioxide gas and water owing to an additional maintenance of the energy and plastic metabolism by exogenous substrates. Such means are usually followed by cucumber and similar brines, coffee, fruit drinks, kvasses and other food products which help in achieving partial detoxication of the organism, enhancing diuresis, restoring hormonal balance. But the deficiency of household means is clearly insufficient.

Widely known is te Alka-Seltzer preparation, one tablet of which comprises 0.324 of acetylsalicylic acid as the active component, 0.965 g of citric acid as an auxiliary ingredient, and 1.625 g of sodium bicarbonate which neutralizes acids with the evolution of carbon dioxide gas on dissolution and provides for an “effersvescence” effect of the resultant drink (source: loose leaf instructions for use of the Alka-Seltzer medicinal preparation manufactured by Miles Corp., Great Britain). This preparation, owing to the effect of the acetylsalicylic acid, inhibits the penetration of toxins into the brain, lowers the intercranial pressure, and mitigates headache. Nevertheless, the Alka-Seltzer preparation does not eliminate the main manifestations of alcohol intoxication and does not restore the capacity for work.

A means of allaying drunkenness is known in the art (RF Patent No. 2012350, IPC: A61 K 35/78, 1994), which comprises 20 drops of an alcoholic peppermint tincture, 2 drops of mint oil, 1 g of succinic acid, 10 g of sugar or fructose, the balance being water. This preparation can only partially eliminate the consequences arising upon intoxication.

A pharmaceutical composition manufactured and sold in Russia, is one of the few products claiming the effect of the prevention and treatment of drunkenness and the alcohol withdrawal syndrome (RF Patent No. 2039556, IPC: A61K 31/19, 1995), and is adopted as the closest functional analog. The formulation of this composition comprises 0.1 to 0.3 g. of succinic acid and 0.025 to 0.085 g of citric acid. This preparation, marketed in Russian Federation under the trade name of Lemontar, can be used under household conditions. In case of alcohol intoxication, it should be taken from 3 to 5 times a day (3 to 5 mg/kg body weight). It provides some adaptogenous, anti-hipoxic and detoxicating effect in 12 to 18 hours after administering the preparation, provided that no alcohol is consumed during that time period.

A conclusion can be drawn that there are presently no recognized medicinal means or food additives that can effectively treat problems associated with alcohol consumption. Similarly, there are currently no medicinal means with the capacity to effectively inhibit the development of these problems prior to, in the course of, or after the consumption of alcohol. In fact, it is still a widely held belief that the reintaking of alcohol (“freshning the nip”) is the most effective remedy available to date. This, however, is a prerequisite step in the process of alcoholism development.

It is an object of the present invention to provide a means for allaying drunkenness, for preventing and removing alcohol intoxication and the hangover syndrome, in the form of a biologically active food additive, which ensures a reduction in the development and liquidation of pathological phenomena of the alcohol withdrawal syndrome, as well as an improvement in alcohol tolerance. Another object of the invention is to provide a method for allaying drunkenness, for preventing and removing alcohol intoxication and the hangover syndrome, which will ensure an effective action of the means prior to, in the course of, and after consumption of alcohol under household conditions, without experienced medical supervision.

Said objects of the invention is accomplished by a means for allaying drunkenness, for preventing and removing alcohol intoxication and the hangover syndrome, which comprises a component based on succinic acid as the active substance, according to the invention, further comprises as active substances at least one component based on L-glutamic acid, sodium mono-L-glutamate, ammonium mono-L-glutamate, ammonium di-L-glutamate, potassium mono-L-glutamate and potassium-di-L-glutamate, at least one component based on fumaric acid, selected from the group consisting of fumaric acid, sodium monofumarate, sodium difumarate, ammonium monofumarate, ammonium difumarate, potassium monofumarate, potassium difumarate, at least one energizer selected from the group consisting of aspartic acid, sodium aspartate, glycine, one of amino acids, sorbic acid, further it comprises additionally ascorbic acid and at least one sugar substitute selected from the group consisting of sucrose, glucose, sorbose, sorbitol, fructose, saccharine, aspartame, xylitol, sorbitol, with the following amount of said components per 1000 mg.

| | |
|---|---|
| Succinic acid | 10 to 400 mg |
| Component based on | 10 to 400 mg |
| L-glutamic acid | |
| Component based on | 2 to 300 mg; |
| fumaric acid | |
| ascorbic acid | 1 to 300 mg |
| sugar substituent and energizer | to make 1000 mg |

As the component based on succinic acid the means a comprise at least one succinic acid salt selected from the group consisting of ammonium monosuccinate, ammonium disuccinate, sodium monosuccinate, sodium disuccinate, potassium monosuccinate, potassium disuccinate, or a mixture of said salts with succinic acid.

To form an effervescent drink, the means further comprises, per 1000 mg of the mixture, a ±20% amount of sodium bicarbonate, equimolar to the amount of the acids. According to the claimed invention, the means is a powder-like mixture or a granulated mixture or a tablet, or a capsule or dragee.

The means can be administered per os or not later than 30 minutes before alcohol consumption, or in the course of alcohol consumption, by swallowing 1-2 tablets or dragees, or capsules, or one glass of the effervescent drink, or after the consumption of alcohol, by swallowing 1 to 4 tablets or dragees, or capsules or 1 to 2 glasses of the effervescent drink.

All the main active components (succinates, fumarates and L-glutamates) are endogenous substrates participating in the main cycle of intracellular energy metabolism—in the Krebs cycle. The auxiliary compound—ascorbic acid—is an additional energizer, and the filler is a sugar substituent, which is a weak energizer too.

All the constituents are well-studied and standard components used in the foodstuffs industry.

Compared with the closest functional analog, (a mixture of succinic acid and citric acid), the proposed means produces a less irritating effect on the mucous membranes of the gastrointestinal tract, has no hypertensive effect, precludes (but not merely stops) the development of hypoxia, ischemias, actively lowers the concentration of ethanol and acetaldehyde in the organism.

Succinates are adaptogens, specific antidotes for acetaldehyde, they are mitochondrial antioxidants, detoxicants, anti-ischemic and anti-hypoxic agents, anti-mutagens and effective energizers.

L-Glutamates are highly effective energizers which effectively enhance the process of ethanol utilization.

Fumarates together with succinates ensure stability of the membranes, stabilize the blood circulation system, providing a powerful, time-extended antihypoxic effect.

Ascorbic acid—vitamic C—is an active water-soluble antioxidant, an activator of the adrenal cortex, this providing an anti-stress effect of the means.

Sugar substitutes, aspartates, amino acids and sorbic acids function as additional energizers. The pharmacokinetics and the time of the aftereffect of the employed substances are different and provide a stable prolonged effect.

It should be noted that the use of the described components separately, even in loading doses, did not lead to an equally tangible effect. The proposed means by the totality of the ingredients comprised therein, owing to their mutual influence, provides a synergistic effect, which manifests itself in a reduction of alcohol intoxication and the prevention or liquidation of the pathological phenomena of the hangover syndrome and in an improvement of alcohol tolerance.

### BRIEF SUMMARY OF THE INVENTION

The proposed means is a biologically active supplement and is a safe food substance. All of its constituents are well-studied and standard components used in the foodstuffs industry. The means efficiently relieves alcohol intoxication partially or completely (depending on the severity of condition). It can also reduce or prevent alcohol withdrawal syndrome, commonly known as hangover. It is preferable to administer the means either before the consumption of alcohol or in the process of consumption in the amount of 0.5 g-1.0 g or after the consumption of alcohol in the amount of 1.0-2.0 g, depending on the volume of consumed alcohol and the severity of condition. No contra-indications were registered. According to genotoxicity trials, the means is an effective anti-mutagenic substance, preventing mutagenesis on the cell and organism levels. The means prevents alcohol pathologies development, since it supports enzyme system of the body. It is an energizing, anti-mutagenic anti anti-hypoxia substance.

### DETAILED DESCRIPTION OF THE INVENTION

Detailed descriptions of the invention are provided herein. It is also to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

According to the claimed invention, the means comprises, as one of the active substances, succinic acid or at least one salt of succinic acid selected from the group consisting of ammonium monosuccinate, ammonium disuccinate, sodium monosuccinate, sodium disuccinate, potassium monosuccinate, potassium disuccinate, or a mixture of said salts with succinic acid. As active substances the means comprises also at least one component based on L-glutamic acid, selected from the group consisting of L-glutamic acid, sodium mono-L-glutamate, sodium di-L-glutamate, ammonium mono-L-glutamate, ammonium di-L-glutamate, potassium mono-L-glutamate, potassium di-L-glutamate, at least one component based on fumaric acid, selected from the group consisting of fumaric acid, sodium monofumarate, sodium difumarate, ammonium monofumarate, ammonium difumarate, potassium monofumarate, potassium difumarate, at least one energizer selected from the group consisting of aspartic acid, sodium aspartate, glycine, one of amino acids, sorbic acid, further, it comprises additionally ascorbic acid and at least one sugar substitute selected from the group consisting of sucrose, glucose, sorbose, sorbitol, fructose, saccharine, aspartame, xylitol, sorbitol, with the following amount of said components per 1000 mg:

| | |
|---|---|
| succinic acid | 10 to 400 mg. |
| a component based on L-glutamic acid of said second | 10 to 400 mg. |
| active substance | |
| a component based on fumaric acid of said third active | 2 to 300 mg. |
| substance | |
| ascorbic acid | 1 to 300 mg. |
| sugar substitute and energizer | to make 1000 mg. |

The means can additionally comprise a 20±% amount of sodium bicarbonate, equimolar to the amount of the comprised acids.

According to the claimed invention, the means is a powder-like mixture or a granulated mixture, or a tablet, or a capsule, or dragee.

The proposed means is prepared by direct mixing of all the components entering into its formulation in dry form, followed by dry tableting pelleting or encapsulating.

The technological process of preparing the proposed means in the form of dragee is carried out in the following sequence: a sugar substitute and ascorbic acid are dissolved in distilled water, evaporated to obtain a highly viscous mass, the salts are compacted to form a pea. This pea is then pelleted in a sugar mass and dried to equilibrium moisture content.

Examples of compositions for preparing the means for allaying drunkenness, preventing and removing alcohol intoxication and the hangover syndrome are presented herein below not to limit the claimed invention in any manner, but merely to illustrate it.

### EXAMPLE 1

A tablet of the proposed means “Antipogmelin” is used, weighing 500 mg, having the following formulation:

100 mg of succinic acid;

100 mg of sodium mono-L-glutaminate;

37.5 mg of fumaric acid;

25 mg of ascorbic acid;

237.5 mg of glucose.

The process for preparing: dry tabletting

### EXAMPLE 2

A powder-like composition is used, weighing 1000 mg, having the following formulation:

200 mg of succinic acid;

200 mg of L-glutamic acid;

75 mg of fumaric acid;

30 mg of aspartic acid;

455 mg of fructose;

440 mg of sodium bicarbonate (for imparting “effervescence”).

The process for preparing: dry mixing

### EXAMPLE 3

A capsule of the means comprises:

400 mg of ammonium monosuccinate;

10 mg of potassium di-L-glutamate

300 mg of sodium monofumarate;

1 mg of ascorbic acid;

50% of sorbitol and 50% of aspartame to make 1000 mg.

The process for preparing: dry mixing followed by encapsulation.

### EXAMPLE 4

A tablet of the means comprises:

400 mg of a mixture of potassium disuccinate (70 mg) and sodium monosuccinate (330 mg);

10 mg of ammonium mono-L-glutamate;

2 mg of potassium difumarate;

300 mg of ascorbic acid;

a mixture of glycine and sodium aspartane taken in equal proportions, to make 100 mg.

The process for preparing: mixing together all the components, except glycine and ammonium nono-L-glutamate, then combined admixing of glycine, the salt and the mixture. The mixture is humidified to 7% by weight, passed through a granulator, and tabletted.

### EXAMPLE 5

The means comprises:

300 mg of sodium disuccinate;

50 mg of a mixture comprising 10 mg of sodium di-L-glutamate and 40 mg of L-glutamic acid;

120 mg of a mixture comprising 20 mg of ammonium; monofumarate and 100 mg of fumaric acid;

200 mg of ascorbic acid;

a mixture of lysine, fructose and sorbic acid, taken in equal proportions, to make 1000 mg.

The process for preparing: dry mixing.

### EXAMPLE 6

The means comprises:

80 mg of a mixture comprising 70 mg of succinic acid and 10 mg of ammonium disuccinate;

250 mg of ascorbic acid;

a mixture of xylitol, sorbose and saccharine, taken in equal proportions, to make 1000 mg.

The process of preparing thereof:

80 mg of mixture comprising 70 mg of succinic acid and 10 mg of ammonium disuccinate, 250 mg of a mixture comprising equal proportions of potassium mono-L-glutamate and of sodium mono-L-glutamate and 160 mg of potassium monofumarate are mixed separately. Ascorbic acid and sucrose substitutes are mixed separately.

The method for allaying drunkenness, for preventing and removing alcohol intoxication and the hangover syndrome is carried out by administering per os the above-described means suitable for use prior to and/or in the course of and/or after intaking alcohol.

Tests in the Examples presented herein below were carried out on groups of volunteers for estimating the proposed compositions in terms of objective and subjective effectiveness.

The test conditions were as follows.

The alcoholic drink was high-quality “Istok” vodka from a single lot. When the intake of alcohol took place under domestic conditions, intaking other alcoholic drinks in accordance with individual preferences was allowable.

The estimates were carried out:

1. when 50 ml of vodka taken on an empty stomach (laboratory conditions);

2. when drinking took place under domestic conditions without limiting the minimum or maximum dose;

3. when arresting the hangover syndrome the next morning after the maximum possible intake of alcohol under domestic conditions.

In all investigations the subjective condition was registered (on the basis of questioning), and a subjective control of the state of the test subjects was carried out by using the parameters of the dynamics of electroencephalographic rhythms, electrocardiography data, volume of the short-term and long-term memory, variational pulsometry and electrocardiography data.

Additional biochemical investigations were carried out for determining the state of carbohydrate metabolism, the degree of anaerobic activation of glycollysis, and of the development of acidosis.

In three volunteers the rate of ethanol oxidation was determined mass-spectosopically from the rate of the 13 C isotope release wit the carbon dioxide of the exhaled air after the taking on an empty stomach 20 ml of vodka added with 40ul of natural ethanol enriched with 13 C isotope.

In the tests “Lemontar” tablets were used (prototype), weighing 250 mg each and comprising 200 mg of succinic acid and 50 mg of citric acid; and the powder-like mixture according to th above-cited Examples 1 and 2, respectively.

Test 1

Under laboratory conditions a group of 12 volunteers of both sexes (5 men and 7 women), who were not used to take alcohol systematically, took 50 ml of vodka. Two kinds of examinations were carried out on an empty stomach:

(a) with a preliminary taking during 30 minutes each kind of the compositions in turn and without taking at random each day (4 tablets of “Lemontar”, 2 tablets of “Antipogmelin”, 3 g of the “effervescent composition” per glass of water”);

(b) with taking, similarly to the above case, directly with vodka or 30 minutes after having taken vodka at will.

Each volunteer took vodka under the laboratory conditions 7 times with an interval of one week: without preparations, with each of the compositions in turn before taking vodka (a), with each of the composition in turn in the course of taking or after taking vodka (b).

As is seen from the data presented in Tables 1 and 2, the claimed means contributed essentially to improving the subjective conditions and the objectively registered parameters of the brain activity, including improvements of the short-term memory, of the cardiovascular system, glucose homeostasis, and ischemic shifts usually caused by alcohol. The revealed positive shifts characterize a marked decrease in the extent of drunkenness when taking alcohol on an empty stomach, in spite of the fact that the rate of ethanol utilization increases sharply both when the proposed means is taken preliminarily and in 30 minutes interval.

Test 2

When vodka was taken under domestic conditions (without dose limitations), the volunteers took compositions (4 tablets of “Lemontar”, 2 tablets of “Antipogmelin”, 3 g of the “effervescent composition” per glass of water) directly before the feast or at the start of it. It was agreed that each of the persons to be examined had to drink at least 300 ml of vodka. Those taking part in the examination were 10 practically healthy men, aged 40-63, who systematically use alcoholic drinks (on an average, 1 or 2 times a week), but have no propensity for alcohol and can lead sober life for a long time.

The observed changes are listed in Tables 4 and 5.

Test 3

The same group as in Example 2, the same conditions of taking alcohol, but taking compositions the next morning (8 tablets of “Lemontar”, 4 tablets of “Antipogmelin” 6 g of the “effervescent composition” per glass of water). The observed changes are presented in Tables 6 and 7.

As is seen from Examples 1-3, the composition of “Antipogmelin” according to Example 1 eliminates most completely the harmful consequences of alcohol consumption. The composition according to Example 2 is slightly inferior in its effect, but more comfortable in taking. Both compositions produce an objectively stronger effect than the “Lemontar” composition adopted as the closest functional analog.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

TABLE 1
Comparative estimation of the subjective condition of volunteers
Enclosed in parentheses is the number of examined subjects (n), who manifested corresponding deviations,
compared with intaking vodka without the composition (time was registered with an accuracy to 1 min.).

| | | Preliminary | Subsequent | Preliminary | Subsequent | Preliminary | Subsequent |
|---|---|---|---|---|---|---|---|
| Symptom being | Registered character- | taking of | taking of | taking of | taking of | taking of “fizzy | taking of |
| compared | istic of the symptom | “Lemontar” | “Lemontar” | “Antipogmelin” | “Antipogmelin” | drink” | “fizzy drink” |
| 1 | 2 | 3 | 4 | 5 | 6 | 7 | 8 |
| Giddiness or any | Time before the | | | | | | |
| symptoms of | appearance: | | | | | | |
| changes in the | Shortening (−2) | +1 (n = 2) | +1 (n = 2) | +2 (n = 4) | +2 (n = 3) | +2 (n = 3) | +2 (n = 2) |
| perception of the | No changes (−1) | −1 (n = 8) | −1 (n = 10) | +1 (n = 6) | +1 (n = 2) | +1 (n = 8) | +1 (n = 8) |
| world around | Prolongation (+1) | −2 (n = 2) | | −1 (n = 2) | −1 (n = 5) | −1 (n = 1) | −1 (n = 2) |
| | Absence of the | | | | | | |
| | symptom (+2) | | | | | | |
| | Duration of manifest- | | | | | | |
| | ation | | | | | | |
| | Shortening (+) | + (n = 3) | + (n = 4) | + (n = 9) | + (n = 10) | + (n = 7) | + (n = 9) 0 |
| | Prolongation (−) | 0 (n − 8) | 0 (n = 6) | 0 (n = 3) | 0 (n = 2) | 0 (n = 5) | (n = 3) |
| | No changes (0) | − (n − 1) | − (n = 2) | | | | |
| Rubbery legs | Time before the | | | | | | |
| | appearance: | | | | | | |
| | Shortening (−2) | +1 (n = 3) | +1 (n = 2) | +2 (n = 3) | +2 (n = 3) | +2 (n = 2) | +2 (n = 3) |
| | No changes (−1) | −1 (n = 9) | 1 (n = 9) | +1 (n = 7) | +1 (n = 3) | +1 (n = 6) | +1 (n = 2) |
| | Prolongation (+1) | | | −1 (n = 2) | −1 (n = 6) | 1 (n = 4) | −1 (n = 7) |
| | Absence of the | | | | | | |
| | symptom (+2) | | | | | | |
| | Duration of manifest- | | | | | | |
| | ation: | | | | | | |
| | Shortening (+) | + (n = 5) | + (n = 3) | + (n = 10) | + (n = 8) | + (n = 8) | + (n = 7) |
| | Prolongation (−) | 0 (n = 5) | 0 (n = 8) | 0 (n = 2) | 0 (n = 4) | 0 (n = 4) | 0 (n = 5) |
| | No changes (0) | − (n − 2) | − (n = 1) | | | | |
| Mood changes | Worsening: aggressive | | | | | | |
| | or | | | | | | |
| | depressive (−) | + (n = 3) | + (n = 3) | + (n = 5) | + (n = 4) | + (n = 5) | + (n = 5) |
| | Improvement: | 0 (n = 7) | 0 (n = 7) | 0 (n = 7) | 0 (n = 8) | 0 (n = 7) | 0 (n = 7) |
| | stable or euphoric (+) | − (n = 2) | − (n = 2) | | | | |
| | No changes (0) | | | | | | |
| Unpleasant | Faster appearance and | | | | | | |
| Sensations | prolonged persistance | | | | | | |
| Epigastral region | (−2) | | | | | | |
| | More short-term, less | +2 (n = 3) | +2 (n = 3) | +2 (n = 5) | +2(n = 5) | +2 (n = 4) | +2 (n = 4) |
| | pronounced (+2) | 0 (n = 6) | 0 (n = 4) | 0 (n = 6) | 0 (n = 5) | 0 (n = 5) | 0 (n = 5) |
| | | − (n = 1) | − (n = 3) | − (n = 1) | − (n = 2) | − (n = 2) | − (n = 3) |
| | Same as without the | −2 (n − 2) | −2 (n = 2) | | | −2(n = 1) | |
| | composition: | | | | | | |
| | absence (0) | | | | | | |
| | presence (−) | | | | | | |
| Headache | Faster appearance and | | | | | | |
| | prolonged persistance | | | | | | |
| | −2) | | | | | | |
| | More short-term, less | +2 (n = 5) | 2 (n = 3) | +2(n = 9) | +2 (n = 8) | +2 (n = 7) | +2 (n = 5) |
| | pronounced (+2) | 0 (n − 6) | 0 (n = 6) | 0 (n = 3) | 0 (n = 4) | 0 (n = 5) | 0 (n = 7) |
| | Same as without the | | | | | | |
| | composition: | | | | | | |
| | absence (0) | | | | | | |
| | presence (−) | | | | | | |
| Any other | Faster appearance and | +2 (n = 4) | +2 (n = 2) | +2 (n = 3) | + (2 n = 3) | +2 (n = 5) | +2 (n = 4) |
| unpleasant | Prolonged persistance | | | | | | |
| sensations | (−2) | | | | | | |
| | More short-term, less | 0 (n = 6) | 0 (n −7) | 0 (n = 8) | 0 (n = 8) | 0 (n = 6) | 0 (n = 6) |
| | pronounced (+2) | −(n = 2) | −(n = 3) | −(n = 1) | −(n = 1) | −(n = 1) | −(n = 2) |
| | Same as without the | | | | | | |
| | composition: | | | | | | |
| | absence (0) | | | | | | |
| | presence (−) | | | | | | |

TABLE 2
Comparative estimation of the subjective condition of volunteers
Enclosed in parentheses is the number of examined subjects (n), who manifested corresponding deviations

| | | | | | Preliminary | Subsequent | | |
|---|---|---|---|---|---|---|---|---|
| | | Intake with- | Preliminary | Subsequent | intake of | intake of | Preliminary | Subsequent |
| Symptom being | Characteristic of | out the | intake of | Intake of | Antipo- | Antipo- | intake of | intake of |
| Compared | symptom | compositionn | “Lemontar” | “Lemontar” | gmelin” | gmelin” | “fizzy drink” | “fizzy drink” |
| 1 | 2 | 3 | 4 | 5 | 6 | 7 | 8 | 9 |
| Disturbance of | Less pronounced | (n = 11) | (n = 10) | (n = 11) | (n = 7) | (n = 9) | (n = 7) | (n = 8) |
| frequency spectrum | alpha- or delta- | 91% | 83% | 91% | 58% | 75% | 58% | 67% |
| one hour later | rhythm | | | | | | | |
| Disturbance of | Arrhythmias (−2) | −2 (n = 5) | −2 (n = 4) | −2 (n = 3) | −2 (n = 2) | −2 (n = 2) | −2 (n = 2) | −2 (n = 2) |
| pulsogram | Change of | −1 (n = 6) | −1 (n = 5) | −1 (n = 6) | −1 (n = 3) | −1 (n = 5) | −1 (n = 4) | −1 (n = 5) |
| during 2 | distribution (−1) | Total 91% | Total 75% | total 75% | total 41% | total 58% | Total 50% | Total 58% |
| hours | No changes (0) | | | | | | | |
| Ischemic | Decrease in voltage, | (n − 5) | (n = 4) | (n = 4) | (n = 2) | (n = 2) | (n = 2) | (n = 3) |
| ECG deviations | Deviation of ST | 41% | 33% | 33% | 16% | 16% | 16% | 25% |
| after 1 hour | interval | | | | | | | |
| Disturbance of | Number of success- | on average | on average | on average | on average | on average | on average | On average |
| short−term | ively reproducible | 3,5 | 4.5 | 4,0 | 5 | 4.5 | 5 | 4,5 |
| memory | cards (norm | 50% | 64% | 57% | 71% | 64% | 71% | 64% |
| | 5 = 71%) | | | | | | | |
| Disturbance of | Number of success- | on average | on average | on average | on average | on average | on average | On average |
| long-term | ively reproducible | 1,5 | 2\hat{ }>> | 2,1 | t | 2,5 | −>> | 2,4 |
| memory | cards (norm | 21% | 33% | 30% | 43% | 36% | 43% | 34% |
| | 3,3 = 47%) | | | | | | | |
| Change of | Increase or sharp | −(n = 6)50% | −(n − 4)33% | −(n = 5)42% | −(n = 3)25% | −(n = 4)33% | −(n = 4)33% | −(n = 4)33% |
| glucose content | drop (−) | +(n = 6) | +(n = 8) | +(n − 7) | +(n = 9) | +(n = 8) | +(n = 8) | +(n = 9) |
| in blood after 1 | Moderate drop or | | | | | | | |
| hour | absence of shift (+) | | | | | | | |
| Change in the | Increase(−) | −(n−9)75% | −(n = 6)50% | −(n = 5)42% | −(n = 4)33% | −(n = 4)33% | −(n = 4)33% | −(n = 4)33% |
| lactate/pyruvate | No changes (0) | 0(n = 3) | 0(n = 5) | 0(n = 7)58% | 0(n − 6)50% | 0(n = 6)50% | 0(n = 6)50% | 0(n = 6)50% |
| ratio after 1 hour | Drop(+) | 25% | 42% | | +(n = 2)17% | +(n = 2)17% | +(n = 2) | +(n − 2)17% |
| | | | +(n = 1)8% | | | | 17% | |
| Presence of Ph | Acidosis or alkalosis | −(n = 9)75% | −(n = 6)50% | −(n = 5)42% | −(n − 4)33% | −(n = 4)33% | −(n = 4)33% | −(n = 4)33% |
| shift | (−) | (n = 3) | +(n = 6)50% | +(n = 7)58% | +(n = 8)67% | +(n = 8)67% | +(n = 8) | +(n = 8)67% |
| | Absence of shift (+) | 25% | | | | | 67% | |

TABLE 3
Characteristic of ethanol metabolism rate judged from ¹³CO₂ release with exhaled air after intaking
compositions and without them (four women aged 38-55 were examined).

| | Intaking vodka | Preliminary | Intake of “Le- | Preliminary | Intake of “Anti- | Preliminary | Intake of “fizzy |
|---|---|---|---|---|---|---|---|
| | without the | Intake of “Le- | Montar” after | Intake of “Anti- | Pogmelin” after | intake of “fizzy | drink” after |
| Registered parameter | composition | montar” | Vodka | pogmelin” | vodka | drink” | vodka |
| 1 | 2 | 3 | 4 | 5 | 6 | 7 | 8 |
| Time of attaining ¹³CO₂ | 90-100 | 85-100 | 80-100 | 60-65 | 80-90 | 65-70 | 70-80 |
| release peak, min. | | | | | | | |
| Full time of ¹³CO₂ | 240-300 | 210-260 | 220-270 | 180-210 | 190-230 | 180-220 | 190-250 |
| release, min. | | | | | | | |
| ¹³CO₂ diminution rate | 7,46 | 8,12 | 7,95 | 9,47 | 9,88 | 9,56 | 9,21 |
| constant (10⁻³) | | | | | | | |
| Duration of plateau in | 60-80 | 45-60 | 45-60 | 30-40 | 30-40 | 30-40 | 30-40 |
| minutes | | | | | | | |
| Peak height, % | 100 | 110 | 110 | 130 | 130 | 130 | 125 |

TABLE 4
Comparative estimation of the subjective condition of volunteers
Enclosed in parentheses is the number of inspected persons (in percent),
who manifested deviations in comparison with usual hangover syndrome after feast

| | Comparative characteristic of | Preliminary | Preliminary | Preliminary |
|---|---|---|---|---|
| Symptom being | the symptom with respect | Intake of | intake of | of intake |
| compared | to usual hangover syndrome | “Lemontar” | “Antipogmelin” | “fizzy drink” |
| 1 | 2 | 3 | 4 | 5 |
| Headache and general | Unusual:: | −2 (20%) | −2 (10%) | |
| perception of the | violent, splitting pain, | −1 (40%) | −1 (30%) | −1 (30%) |
| world around | heavy head, hampered perception (−1) | 0 (20%) | 0 (30%) | 0 (40%) |
| | as usual (0) | +2 (20%) | +2 (30%) | +2 (30%) |
| | clear perception (+2) | | | |
| Unpleasant taste in the | Less than usual (+) | + (50%) | + (70%) | + (70%) |
| mouth, furred tongue | worse than usual (−) | 0 (40%) | 0 (30%) | 0 (30%) |
| | as usual (0) | − (10%) | | |
| General condition | Unusual sensation: | + (30 | + (70%) | + (60%) |
| | Sensation of breakdown (−) | 0 (70%) | 0 (30%) | 0 (40%) |
| | renewed energy (+) | | | |
| | as usual (0) | | | |
| Change of mood | Worsening:: | − (10%) | − (10%) | |
| | Aggressive or depressive (−) | + (40%) | + (60%) | + (50%) |
| | Improvement: stable or euphoric (+) | 0 (50%) | 0 (30%) | 0 (50%) |
| | As usual (0) | | | |
| Unpleasant sensations | Unusual manifestation of: | −2 (10%) | − (10%) | − (10%) |
| from the side of | acute pain, nausea, vomiting (−2) | − (10%) | + (60%) | + (60%) |
| gastrointestinal tract | painfulness, nauseating feeling or | + (40%) 0 | 0 (30%) | 0 (30%) |
| | lowering of appetite (−1) | (40%) | | |
| | No deviations from usual hangover (0) | | | |
| | Improvement of appetite (+) | | | |
| Any other unpleasant | Appearance and prolonged persistance (−2) | − (10%) | + (60%) | + (60%) |
| sensations | short-term, unexpressed (−1) | + (50%) | 0 (40%) | 0 (40%) |
| | absence (+) | 0 (40%) | | |
| | as usual (0) | | | |

TABLE 5
Comparative estimation of the subjective condition of volunteers
Enclosed in parentheses is the number of inspected persons who manifested deviations

| | Characteristic of symptom | Kind and/or percentage of deviations | | |
|---|---|---|---|---|
| | and indication of | Taking | Taking “Anti- | Taking “fizzy |
| Symptom being compared | Normal response | “Lemontar” | pogmelin” | drink” |
| 1 | 2 | 3 | 4 | 5 |
| Disturbance of EEC frequency | Less pronounced alpha or delta rhythm | 60% | 40% | 40% |
| spectrum next morning | (usually at least 75%) | | | |
| Disturbance of pulsogram during | Arrhythmias (−2) usually 90% | −2 (50%) −1 | −2 (10%) −1 | −2 (20%) −1 |
| 2 hours in the morning | Change of distribution (−1) usually to 100% | (50%) | (60%) | (60%) |
| | No changes (0) usually to 1-2% | | 0 (30%) | 0 (20%) |
| Ischemic deviations of ECG | Change of voltage, deviation of ST interval | 40% | 30% | 30% |
| next morning | (usually 50 to 70%) | | | |
| Volume of short-term memory | Number and sequence of reproducible cards | 41% | 49% | 46% |
| during investigation | out of 7 (norm is 70%; in case of hangover, | | | |
| | not over 35%) | | | |
| Volume of long-term memory, | Number and sequence of reproducible cards | 19% | 27% | 26% |
| checking next day | out of 7 (norm is 45−47%; in case of | | | |
| | hangover, not over 15%) | | | |
| Change in glucose content in | Increase or sharp drop, usually 70-80% | 60% | 40% | 40% |
| blood next day | | | | |
| Change in lactate/pyruvate ratio | Increase (−), usually up to 100% | 0 (30%)-(70%) | 0 (50%) | 0 (50%) |
| next day | No changes (0) | | − (50%) | − (50%) |
| | Drop (+) | | | |
| Presence of pH shift next day | Acidosis or alkalosis (−), usually up to 100% | 0 (40%)-(60%) | 0 (50%)-(50%) | 0 (50%) |
| | Absence of shift (+) | | | − (50%) |

TABLE 6
Comparative estimation of the subjective condition of volunteers
Enclosed in parentheses is the number of inspected persons (in percent),
who manifested deviations in comparison with usual hangover syndrome after feast

| | | Taking | Taking | Taking |
|---|---|---|---|---|
| Symptom being | Comparative characteristic of the symptom | “Lemontar” | “Antipogmelin” | “fizzy drink” |
| compared | with respect to usual hangover syndrome | next morning | next morning | next morning |
| 1 | 2 | 3 | 4 | 5 |
| Headache and general | Unusual:: | −2 (10%) | | |
| perception of the | violent, splitting pain, | −1 (20%) | | −1 (10%) |
| world around | heavy head, hampered perception (−1) | 0 (40%) | 0 (30%) | 0 (30%) |
| | as usual (0) | +2 (30%) | +2 (70%) | +2 (60%) |
| | clear perception (+2) | | | |
| General condition | Unusual sensation: | + (30%) | + (70%) | + (70%) |
| | Sensation of breakdown (−) | 0 (70%) | 0 (30%) | 0 (30%) |
| | renewed energy (+) | | | |
| | as usual (0) | | | |
| Change of mood | Worsening:: | − (10%) | − (10%) | |
| | Aggressive or depressive (−) | + (40%) | + (60%) | + (50%) |
| | Improvement: stable or euphoric (+) | 0 (50%) | 0 (30%) | 0 (50%) |
| | As usual (0) | | | |
| Unpleasant sensations | Unusual manifestation of: | − (20%) | | − (10%) |
| from the side of | acute pain, nausea, vomiting (−2) | + (40%) | + (60%) | + (60%) |
| gastrointestinal tract | Painfulness, nauseating feeling | 0 (40%) | 0 (40%) | 0 (50%) |
| | or lowering of appetite (−1) | | | |
| | Absence of deviation from usual hangover (0) | | | |
| | Improvement of appetite (+) | | | |
| Any other unpleasant | Appearance and prolonged persistence (−2) | − (10%) | + (60%) | + (60%) |
| sensations | short-term, not pronounced (−1) | + (50%) | 0 (40%) | 0 (40%) |
| | Absence (+) | 0 (40%) | | |
| | As usual (0) | | | |

TABLE 7
Comparative estimation of the subjective condition of volunteers
Enclosed in parentheses is the number of examined subjects who manifested corresponding deviations

| | | Kind and/or percentage of deviations | | |
|---|---|---|---|---|
| | Characteristic of symptom | Taking | Taking “Anti- | Taking “fizzy |
| | and indication of | “Lemontar” | pogmelin” | drink” |
| Symptom being compared | normal response | next morning | next morning | next morning |
| 1 | 2 | 3 | 4 | 5 |
| Dynamics of EEG spectrum | Suppression of alpha or delta rhythm (−) | + (40)% | + (60)% | + (60)% |
| next morning | Improved manifestation of alpha or delta | 0 (40%) | 0 (40%) | 0 (40%) |
| | Rhythm (+) | − (20%) | | |
| | No changes (0) | | | |
| Disturbance of pulsogram during | Arrhythmias (−), usually 90% | − (20%) | | |
| two hours in the morning | Improved distribution, antiarrhythmic | + (40%) | + (60%) | + (60%) |
| | effect (+) | 0 (40%) | 0 (40%) | 0 (40%) |
| | No changes (0) | | | |
| Ischemic ECG deviations next | Worsening (−) | − (30%) | | |
| morning | Improvement (+) | + (20%) | + (40%) | + (40%) |
| | No changes (0) | 0 (60%) | 0 (60%) | 0 (60%) |
| Volume of short-range memory | Worsening (−) | − (10%) | | |
| during investigations | Improvement (+) | + (20%) 0 (70%) | + (50%) | + (40%) |
| | No changes (0) | | 0 (50%) | 0 (60%) |
| Change of glucose content in | Increase or sharp drop (−) | − (30%) | | |
| blood next day | Small drop (+) | + (20%) | + (30%) | + (30%) |
| | No changes (0) | 0 (50%) | 0 (70%) | 0 (70%) |
| Change of lactate/pyruvate | Increase (−), usually up to 100% | − (30%) | | |
| ratio next day | No changes (0) | + (20%) | + (30%) | + (30%) |
| | Drop (+) | 0 (50%) | 0 (70%) | 0 (70%) |
| Presence of pH shift next day | Acidosis (−) | 0 (40%) | 0 (50%) | 0 (50%) |
| | Alkalosis (+) | + (50%) | + (50%) | + (50%) |
| | No shift (0) | − (10%) | | |

What is claimed is:

1. A means for allaying drunkenness, preventing and removing alcohol intoxication and hangover syndrome and a method for allaying drunkenness, preventing and removing alcohol intoxication and hangover syndrome by using this means, Comprising:
a first active substance which comprises a component based on succinic acid;
a second active substance which comprises at least one component based on L-glutamic acid, selected from the group consisting of L-glutamic acid, sodium mono-L-glutamate, sodium di-L-glutamate, ammonium mono-L-glutamate, ammonium di-L-glutamate, potassium mono-L-glutamate, potassium di-L-glutamate,
a third active substance comprising at least one component based on fumaric acid, selected from the group consisting of fumaric acid, sodium fumarate, sodium difumarate, ammonium monofumarate, ammonium difumarate, potassium monofumarate, potassium difumarate;
ascorbic acid;
at least one energizer selected from the group consisting of aspartic acid, sodium aspartate, glycine, one of amino acids, sorbic acid;
at least one sugar substitute selected from the group consisting of sucrose, glucose, sorbose, sorbitol, fructose, saccharine, aspartame, xylitol, sorbitol,
said first, second, third substances, ascorbic acid, at least one energizer and at least one sugar substitute taken in the following amount per 1000 mg:

| | |
|---|---|
| a component based on succinic acid | 10 to 400 mg. |
| of said first active substance | |
| a component based on L-glutamic acid | 10 to 400 mg. |
| of said second active substance | |
| a component based on fumaric acid | 2 to 300 mg. |
| of said third active substance | |
| ascorbic acid | 1 to 300 mg. |
| sugar substitute and energizer | to make 1000 mg. |

2. A means according to claim 1, which comprises as said component based on succinic acid at least one salt of succinic acid, selected from the group consisting of ammonium monosuccinate, ammonium disuccinate, sodium monosuccinate, sodium disuccinate, potassium monosuccinate, potassium disuccinate or a mixture thereof with succinic acid.

3. A means according to claim 1, which further comprises a +20% amount of sodium bicarbonate equimolar to the amount of said acids.

4. A means according to claim 2, which further comprises a +20% amount of sodium bicarbonate equimolar to the amount of said acids.

5. A means according to claim 1, which is a powder-like mixture.

6. A means according to claim 1, which is a granulated mixture.

7. A means according to claim 1, which is a tablet.

8. A means according to claim 1, which is capsule.

9. A means according to claim 1, which is a dragee.

10. A means according to claim 3, which is an effervescent drink.

11. A means according to claim 4, which is an effervescent drink.

12. A method for allaying drunkenness, for preventing and removing alcohol intoxication and the hangover syndrome, consisting in administering a medicinal means comprising:
a first active substance which comprises a component based on succinic acid;
a second active substance which comprises at least one component based on L-glutamic acid, selected from the group consisting of L-glutamic acid, sodium mono-L-glutamate, sodium di-L-glutamate, ammonium mono-L-glutamate, ammonium di-L-glutamate, potassium mono-L-glutamate, potassium di-L-glutamate,
a third active substance comprising at least one component based on fumaric acid, selected from the group consisting of fumaric acid, sodium fumarate, sodium difumarate, ammonium monofumarate, ammonium difumarate, potassium monofumarate, potassium difumarate;
ascorbic acid;
at least one energizer selected from the group consisting of aspartic acid, sodium aspartate, glycine, one of amino acids, sorbic acid;
at least one sugar substitute selected from the group consisting of sucrose, glucose, sorbose, sorbitol, fructose, saccharine, aspartame, xylitol, sorbitol,
said first, second, third substances, ascorbic acid, at least one energizer and at least one sugar substitute taken in the following amount per 1000 mg:

| | |
|---|---|
| of said first active substance | 10 to 400 mg. |
| a component based on L-glutamic acid | 10 to 400 mg. |
| of said second active substance | |
| a component based on fumaric acid | 2 to 300 mg. |
| of said third active substance | |
| ascorbic acid | 1 to 300 mg. |
| sugar substitute and energizer | to make 1000 mg. |

13. A method according to claim 12 , wherein said medicinal means is administered prior to intaking alcohol.

14. A method according to claim 12, wherein said medicinal means is administered in the course of intaking alcohol.

15. A method according to claim 12, wherein said medicinal means is administered after intaking alcohol.

16. A method according to claim 12, wherein said medicinal means is administered prior to and in the course of intaking alcohol.

17. A method according to claim 12, wherein said medicinal means is administered prior to and after intaking alcohol.

18. A method according to claim 12, wherein said medicinal means is administered in the course of and after intaking alcohol.

19. A method according to claim 12, wherein as said component based on succinic acid use is made of at least one salt of succinic acid, selected from the group consisting of ammonium monosuccinate, ammonium disuccinate, sodium monosuccinate, ammonium disuccinate, potassium monosuccinate, potassium disuccinate or a mixture thereof with succinic acid.

20. A method according to claim 12, wherein use is additionally made of a +20% amount of sodium bicarbonate equimolar to the amount of said acids.

21. A method according to claim 19, wherein use is additionally made of a +20% amount of sodium bicarbonate equimolar to the amount of said acids.

22. A method according to claim 12, wherein said medicinal means is used in the form of a powder-like mixture.

23. A method according to claim 12, wherein said medicinal means is used in the form of a granulated mixture.

24. A method according to claim 12, wherein said medicinal means is used in the form of a tablet.

25. A method according to claim 12, wherein said medicinal means is used in the form of a dragee.

26. A method according to claim 12, wherein said medicinal means is used in the form of a capsule.

27. A method according to claim 24, wherein said medicinal means in the form of a tablet is administered per os in an amount of 1 or 2 tablets not later than 30 minutes prior to intaking alcohol.

28. A method according to claim 24, wherein said medicinal means in the form of a tablet is administered per os in an amount of 1 or 2 tablets in the course of intaking alcohol.

29. A method according to claim 24, wherein said medicinal means in the form of a tablet is administered per os in an amount of 1 or 4 tablets after intaking alcohol.

30. A method according to claim 25, wherein said medicinal preparation in the form of dragee is administered per os in an amount of 1 or 2 dragees not later than 30 minutes prior to intaking alcohol.

31. A method according to claim 25, wherein said medicinal preparation in the form of dragee is administered per os in an amount of 1 or 2 dragees in the course of intaking alcohol.

32. A method according to claim 25, wherein said medicinal preparation in the form of dragee is administered per os in an amount of 1 or 4 dragees after intaking alcohol.

33. A method according to claim 26, wherein said medicinal means in the form of a capsule is administered per os in an amount of 1 to 2 capsules not later than 30 minutes prior to intaking alcohol.

34. A method according to claim 26, wherein said medicinal means in the form of a capsule is administered per os in an amount of 1 to 2 capsules in the course of intaking alcohol.

35. A method according to claim 26, wherein said medicinal means in the form of a capsule is administered per os in an amount of 1 to 4 capsules after intaking alcohol.

36. A method according to claim 2, wherein said medicinal means is used in the form of an effervescent drink.

37. A method according to claim 36, wherein said medicinal means in the form of an effervescent drink is administered per os in an amount of one glass not later than 30 minutes prior to intaking alcohol.

38. A method according to claim 36, wherein said medicinal means in the form of an effervescent drink is administered per os in an amount of one glass in the course of intaking alcohol.

39. A method according to claim 36, wherein said medicinal means in the form of an effervescent drink is administered per os in an amount of 1 to 2 glasses after intaking alcohol.
